(19) United States
(12) Patent Application Publication
(10) Pub. No.: US 2003/0053961 A1
(43) Pub. Date: Mar. 20, 2003

(54) Mousse forming compositions comprising quaternary ammonium agents

(76) Inventor: Wayne Ellis Eccard, Cleves, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/193,805

(22) Filed: Jul. 12, 2002

Related U.S. Application Data
(60) Provisional application No. 60/305,597, filed on Jul. 13, 2001.

(51) Int. Cl. A61K 7/075; A61K 7/08; A61K 9/00

(52) U.S. Cl. 424/47; 424/70.28

(57) ABSTRACT

The present invention is directed to a mousse-forming cosmetic composition, comprising by weight of said composition (i) at least 0.1% of a quaternary ammonium agent capable of forming vesicles, (ii) at least 1% of emollient. The composition also preferably further comprises at least 1% of humectant.

### CROSS REFERENCE TO RELATED APPLICATION

This application claims the benefit to U.S. Application Serial No. 60/305,597 filed Jul. 13, 2001.

### TECHNICAL FIELD

The present invention is directed to a mousse-forming cosmetic composition. More particularly, this invention is directed to a cosmetic composition comprising a quaternary ammonium agent and an oil-soluble skin-conditioning agent such as an emollient. The composition provides good moisturisation, hydration, skin feel, skin softness and skin smoothness benefits and can be used for conditioning the whole of the body.

### BACKGROUND

Skin is made up of several layers of cells that coat and protect the keratin and collagen fibrous proteins that form the skeleton of its structure. The outermost of these layers, referred to as the stratum corneum, is known to be composed of 25 nm protein bundles surrounded by 8 nm thick layers. Anionic surfactants and organic solvents typically penetrate the stratum corneum membrane and, by delipidization (i.e. removal of the lipids from the stratum corneum), destroy its integrity. This destruction of the skin surface topography leads to a rough feel and may eventually permit the surfactant or solvent to interact with the keratin, creating irritation. Many people expose their skin to this type of insult every day. In addition, the skin can be damaged by other factors such as exposure to cold air or wind, mechanical abrasion, immersion in water, ageing, etc. Thus, there exists a need for a way of mitigating or ameliorating this damage.

Skin care compositions aiming at improving the stratum corneum have been commonly used and usually comprise at least one humectant and/or at least one emollient. Humectants, or moisturizing agents, are cosmetic ingredients with water binding properties that are capable of retaining large amounts of water relative to their weight. Humectants are usually more soluble in water than in oil.

Emollients such as polyol carboxylic acid esters are cosmetic ingredients which help to maintain the soft, smooth, and pliable appearance of the skin. Emollients function by their ability to remain on the skin surface or in the stratum corneum to act as lubricants, to reduce flaking, and to improve the skin's appearance. Whereas humectants are considered to add water to the skin, emollients hydrate the skin by occlusion and thereby conserve the water content of the stratum corneum. Emollients are usually less soluble in water than in oil.

These agents are however usually associated with negatives in terms of poor skin-feel. They often feel very greasy on the skin and have poor rub-in, absorption and residue characteristics. Considerable attention has been given in the past to find new ways of delivering these skin benefit agents without suffering from undesirable negatives. Quaternary ammonium agents capable of forming vesicular systems have been used in creams and lotions to efficiently deliver high levels of emollients and humectants to the skin with reduced negatives in terms of poor skin feel.

While the prior art cosmetic compositions containing quaternary ammonium agents have addressed some of the negatives, they have not addressed the problems to the extent of the present invention. Therefore, there is a need for an improved cosmetic composition.

### SUMMARY

The present invention is directed to a mousse-forming cosmetic composition characterized in that it comprises by weight of the composition:

(i) at least 0.1% of a quaternary ammonium agent;

(ii) at least 1% of an oil-soluble skin conditioning agent, preferably an emollient.

The composition preferably further comprises at least 1% by weight of water-soluble skin conditioning agent (preferably a humectant). The quaternary ammonium agent is preferably capable of forming vesicles.

A concentrate and a propellant packaged in a pressurized container can be used to dispense the mousse-forming composition.

The mousse-forming composition is preferably used as a rinse-off composition for skin or hair.

### DETAILED DESCRIPTION

While the specification concludes with claims that particularly point out and distinctly claim the invention, it is believed the present invention will be better understood from the following description.

All cited references are incorporated herein by reference in their entireties. Citation of any reference is not an admission regarding any determination as to its availability as prior art to the claimed invention.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “mousse”, as used herein, refers to the dispensed product unless otherwise specified.

The term “a composition substantially free of X” as used herein means that the composition comprises, by weight, less than about 5%, preferably less than about 2%, more preferably less than about 0.5%, even more preferably less than about 0.1% of X.

All percentages are by weight of the composition unless specifically stated otherwise. When the composition comprises a propellant, its percentage is expressed by weight of the total composition, which is the sum of the weight of the concentrate and of the weight of the propellant.

The term “water-soluble skin conditioning agent”, as used herein, refers to skin conditioning agents having a higher affinity for the aqueous phase of an emulsion than for the oil phase. The term “oil-soluble skin-conditioning agent”, as used herein, refers to skin conditioning agents having a higher affinity for the oil phase than for the aqueous phase of an emulsion. A list of commonly used skin conditioning agents can be found in CTFA International Cosmetic Ingredient Dictionary and Handbook, 8th edition, edited by Wenninger and Canterbery (The Cosmetic, Toiletry, and Fragrance Association, Inc., Washington, D.C., 2000), p. 1767-1785.

Preferably, the non-gaseous phase of the mousse is in the form of an oil-in-water emulsion of one or more oil phases in an aqueous continuous phase, each oil phase comprising a single oily component or a mixture of oily components in miscible or homogeneous form but said different oil phases containing different materials or combinations of materials from each other.

The compositions of the present invention preferably comprise vesicles formed by a quaternary ammonium agent. As used herein the term “vesicle” means one or more bilayers arranged in a closed, usually spherical geometry, said bilayer comprises quaternary ammonium agent as described hereinbelow.

The compositions of the present invention are preferably substantially free of anionic and non-ionic surfactant. Anionic surfactants should preferably be avoided because they could interact with the positively charged quaternary ammonium compounds and hinder the formation of vesicles. Non-ionic surfactants (for example condensation products of alkylene oxides with acids, alcohol or both) tend to remove skin-conditioning ingredients such as emollients and humectants from the skin.

In a preferred embodiment of the present invention, the mousse is formed by the passage of a pressurized mixture of a concentrate and a propellant through a nozzle. The term “concentrate” will be used throughout this specification to refer to the liquid contents of the dispenser, other than the propellant; “liquid” in this context embracing solutions, emulsions and suspensions. In other words, the concentrate itself may be an emulsion, suspension or solution. In this context, the concentrate used to provide the mousse of the present invention is preferably formulated so as to have a product viscosity of at least about 1,000 mPa.s and preferably in the range from about 1,000 to about 300,000 mPa.s, more preferably from about 1,000 to about 250,000 mPa.s and especially from about 1,000 to about 200,000 mPa.s (26.8° C., neat, Brookfield DV-II+ Spindle CP52/CP41).

Once dispensed, the mousse composition comprises negligible amount of propellant. The concentrate therefore comprises, by weight,

(i) at least 0.1% of a quaternary ammonium agent;

(ii) at least 1% of oil-soluble skin conditioning agent, and, where included,

(iii) at least 1% of water-soluble skin conditioning agent.

It has now been unexpectedly found that delivering cosmetic compositions comprising an oil-soluble skin conditioning agent such as an emollient and quaternary ammonium agents in the form of a mousse (for example by addition of a propellant) results in the formation of an extremely stable mousse and improved skin care benefits.

Whilst not wishing to be bound by theory, it is believed that the quaternary ammonium agents act as emulsifiers and stabilize the oil droplets (which comprise the majority of the emollient) within the pre-dispensed mousse. When the mousse is dispensed, some of the quaternary ammonium agent molecules migrate to the air/water interface of the mousse, thereby maintaining a stable foam. The reduction of the concentration of quaternary ammonium agents within the bulk of the compositions results in the destabilization of oil droplets, leading to droplet coalescence. This increase in droplet size drives enhanced deposition from the mousse form, leading to improved skin benefits.

The composition preferably further comprises a humectant. The quaternary ammonium agents are preferably capable of forming vesicles that can trap water-soluble skin-conditioning agents such as humectants. After the mousse is dispensed, the vesicles deposit on the skin and protect the emollient from being immediately rinsed away by water. It is believed that the vesicles are sufficiently stable to remain unaffected by the migration of the quaternary ammonium agent to the air-mousse interface.

Humectants and emollients are preferred skin-conditioning agents for use in the present invention, however any water-soluble skin-conditioning compound and oil-soluble-skin conditioning compound can be efficiently delivered together to the substrate.

### Quaternary Ammonium Agent

The compositions of the present invention must comprise at least one quaternary ammonium agent suitable for use in cosmetic compositions. The expression “quaternary ammonium agent” as used herein encompasses quaternary ammonium compounds, protonated tertiary amines and mixtures thereof. The expression “quaternary ammonium agent” includes a compound or mixture of compounds having a quaternary nitrogen atom substituted with one or more, preferably two, moieties containing six or more carbon atoms. Preferably the quaternary ammonium agents for use herein are selected from those having a quaternary nitrogen substituted with two moieties wherein each moiety comprises ten or more, preferably 12 or more, carbon atoms. Particularly preferred quaternary ammonium agents for use herein are selected from those which are able to form vesicles in polar solvents, as detected by microscopic analysis (polarized light microscopy at a magnification of x60 using a Nikon Eclipse E800 microscope).

The present compositions comprise at least about 0.1%, preferably at least about 0.5%, more preferably at least about 1.5%, even more preferably at least about 3%, by weight, of a quaternary ammonium agent.

Preferably the quaternary ammonium agents for use herein are selected from:

(a) quaternary ammonium compounds according to general formula (I):

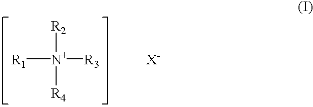

wherein, R₁ & R₂ are each C₁-C₄ alkyl or C₁-C₄ hydroxyalkyl groups or hydrogen. R₃ & R₄ are each alkyl or alkenyl groups having from about 8 to about 22 carbon atoms. X⁻ is a salt forming anion, compatible with quaternary ammonium compounds and other adjunct ingredients.

Preferred quaternary ammonium compounds of this type are quaternised amines having the general formula (I) where R₁ & R₂ are methyl or hydroxyethyl and R₃ & R₄ are linear or branched alkyl or alkenyl chains comprising at least 11 atoms, preferably at least 15 carbon atoms.

(b) quaternary ammonium compounds according to general formula (II) or (III):

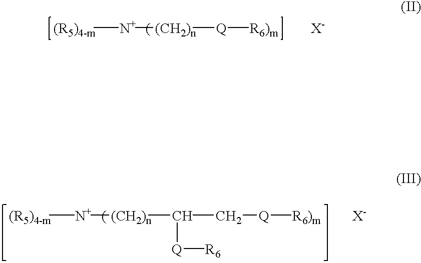

wherein each R₅ unit is independently selected from hydrogen, branched or straight chain C₁-C₆ alkyl, branched or straight chain C₁-C₆ hydroxyalkyl and mixtures thereof, preferably said R₅ unit is methyl or hydroxyethyl; wherein each R₆ unit is selected from the group consisting essentially of independently linear or branched C₁₁-C₂₂ alkyl, linear or branched C₁₁-C₂₂ alkenyl, and mixtures thereof; wherein X⁻ is an anion which is compatible with skin care actives and adjunct ingredients; wherein m is from 1 to 4, preferably 2; wherein n is from 1 to 4, preferably 2 and Q is a carbonyl unit selected from

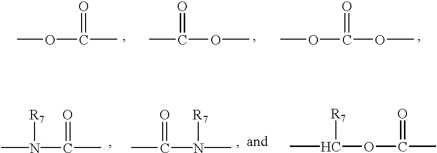

wherein R₇ is hydrogen, C₁-C₄ alkyl, C₁-C₄ hydroxyalkyl, and mixtures thereof.

In the above quaternary ammonium compound example, the unit -QR₆ contains a fatty acyl unit which is typically derived from a triglyceride source. The triglyceride source is preferably derived from tallow, partially hydrogenated tallow, lard, partially hydrogenated lard, vegetable oils and/or partially hydrogenated vegetable oils, such as, canola oil, safflower oil, peanut oil, rapeseed oil, sunflower oil, corn oil, soybean oil, tall oil, rice bran oil, etc. and mixtures of these oils.

The R6 units are typically mixtures of linear and branched chains of both saturated and unsaturated aliphatic fatty acids, an example of which (canola oil), is described in Table I herein below.

TABLE I

| Fatty acyl unit | % |
|---|---|
| C14 | 0.1 |
| C16 | 5.4 |
| C16:1 | 0.4 |
| C18 | 5.7 |
| C18:1 | 67 |
| C18:2 | 13.5 |
| C18:3 | 2.7 |
| C20 | 0.5 |
| C20:1 | 4.6 |

The counterion, X⁻ in the above compounds, can be any compatible anion, preferably the anion of a strong acid, for example, chloride, bromide, methylsulfate, ethylsulfate, sulfate, nitrate and the like, more preferably chloride or methyl sulfate. The anion can also, but less preferably, carry a double charge in which case X⁻ represents half a group.

The preferred quaternary ammonium compounds of the present invention are the Diester and/or Diamide Quaternary Ammonium (DEQA) compounds, the diesters and diamides having general formula (II), wherein the carbonyl group Q is selected from:

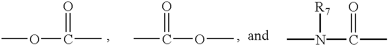

Tallow, canola and palm oil are convenient and inexpensive sources of fatty acyl units which are suitable for use in the present invention as QR₆ units.

The counterion, X⁻, can be chloride, bromide, methylsulfate, formate, sulfate, nitrate, and mixtures thereof. In fact, the anion, X, is merely present as a counterion of the positively charged quaternary ammonium compounds. The scope of this invention is not considered limited to any particular anion.

As used herein, when the diester is specified, it will include the monoester and triester that are normally present as a result of the manufacture process.

(c) protonated tertiary amine compounds according to general formula (IV) or (V):

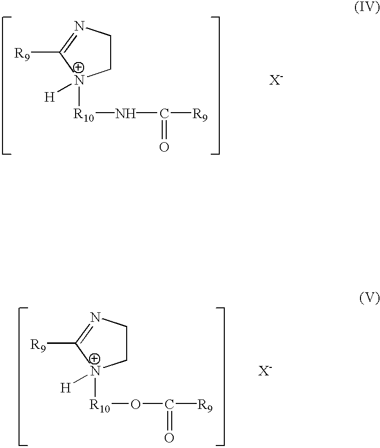

wherein R₉ is an acyclic aliphatic C₁₅-C₂, hydrocarbon group and R₁₀ is a C₁-C₆ alkyl or alkylene group.

These ammonium compounds, having a pKa value of not greater than about 4, are able to generate a cationic charge in situ when dispersed in an aqueous solution, providing that the pH of the final composition is not greater than about 6.

d) quaternary ammonium compounds according to general formula (VI) or (VII):

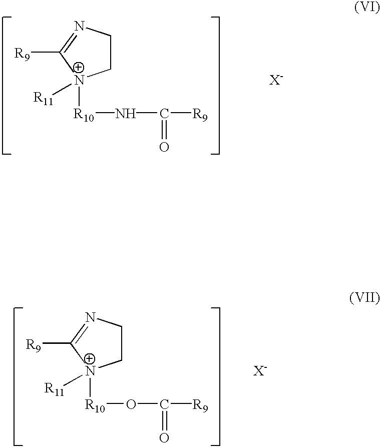

wherein R₉ & R₁₀ are as specified hereinabove and R₁₁ is selected from C₁-C₄ alkyl and hydroxyalkyl groups.

The counterion, X⁻, can be chloride, bromide, methylsulfate, formate, sulfate, nitrate, and mixtures thereof. In fact, the anion, X, is merely present as a counterion of the positively charged quaternary ammonium compounds. The scope of this invention is not considered limited to any particular anion.

(e) Quaternary ammonium compounds and protonated tertiary amines according to general formula (VII) or (IX):

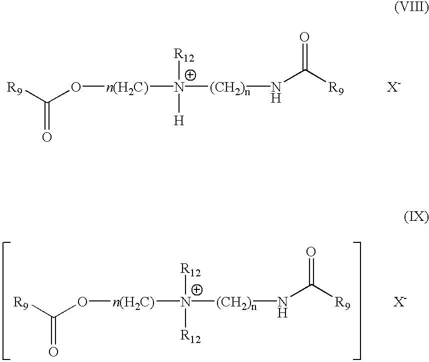

wherein, n is from 1 to 6, R₉ is selected from acyclic aliphatic C₁₅-C₂₁ hydrocarbon groups and R₁₂ is selected from C₁-C₄ alkyl and hydroxyalkyl groups.

These ammonium compounds (VII), having a pKa value of not greater than about 4, are able to generate a cationic charge in situ when dispersed in an aqueous solution, providing that the pH of the final composition is not greater than about 6.

The counterion, X⁻ (IX), can be chloride, bromide, methylsulfate, formate, sulfate, nitrate, and mixtures thereof. In fact, the anion, X, is merely present as a counterion of the positively charged quaternary ammonium compounds. The scope of this invention is not considered limited to any particular anion.

(f) diquaternary ammonium compounds according to general formula (X), (XI), (XII) or (XIII):

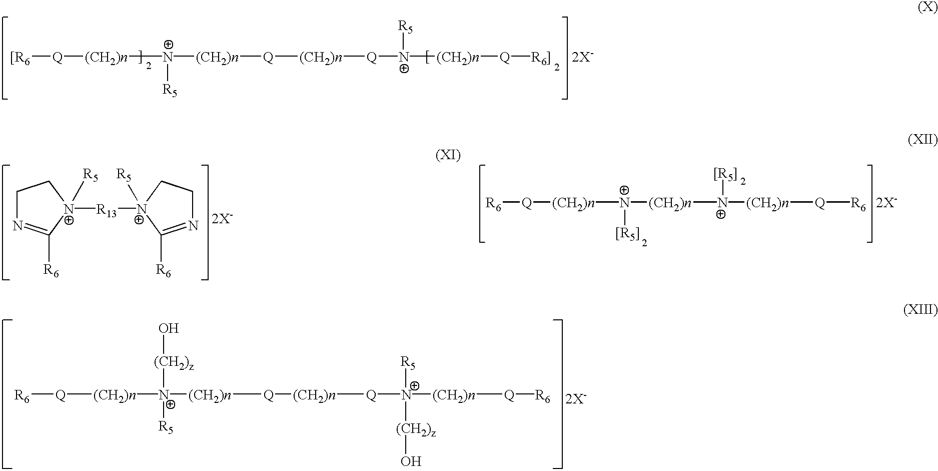

wherein R₅, R₆, Q, n & X⁻ are as defined hereinabove in relation to general formula (II) and (III), R₁₃ is selected from C₁-C₆ alkylene groups, preferably an ethylene group and z is from 0 to 4.

(g) mixtures of the above quaternary ammonium compounds.

The preferred quaternary ammonium agents for use in the present invention are those described in section (b) hereinabove. In particular, diester and/or diamide quaternary ammonium (DEQA) compounds according to general formula (II) hereinabove are preferred. Preferred diesters for use herein are those according to general formula (II) wherein R₅, R₆, and X⁻ are as defined hereinabove and Q is:

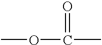

Preferred diamides for use herein are those according to general formula (II) wherein R₅, R₆, and X⁻ are as defined hereinabove and Q is:

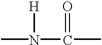

Preferred examples of quaternary ammonium compounds suitable for use in the compositions of the present invention are N,N-di(canoloyloxyethyl)-N,N-dimethylammonium chloride, N,N-di(canoloyloxyethyl)-N-methyl-N-(2-hydroxyethyl)ammonium methyl sulfate, N,N-di(canoloyloxyethyl)-N-methyl-N-(2-hydroxyethyl)ammonium chloride and mixtures thereof. Particularly preferred for use herein is N,N-di(canoloyloxyethyl)-N-methyl-N-(2-hydroxyethyl)ammonium methyl sulfate.

Although quaternary ammonium compounds are derived from “canoloyl” fatty acyl groups are preferred, other suitable examples of quaternary ammonium compounds are derived from fatty acyl groups wherein the term “canoloyl” in the above examples is replaced by the terms “tallowoyl, cocoyl, palmoyl, lauroyl, oleoyl, ricinoleyl, stearoyl, palmitoyl” which correspond to the triglyceride source from which the fatty acyl units are derived. These alternative fatty acyl sources can comprise either fully saturated, or preferably at least partly unsaturated chains.

### Oil-Soluble Skin-Conditioning Agent

A second essential element of the compositions of the present invention is that they comprise at least 1%, by weight, of an oil-soluble skin-conditioning agent. Preferred oil-soluble skin-conditioning agents are emollients. Emollients tend to lubricate the skin, increase the smoothness and suppleness of the skin, prevent or relieve dryness of the skin, and/or protect the skin. A wide variety of suitable emollients are known and may be used herein. Sagarin, Cosmetics, Science and Technology, 2nd Edition, Vol. 1, pp. 32-43 (1972) contains numerous examples of materials suitable for use as emollients. Another list of suitable emollient can be found in CTFA International Cosmetic Ingredient Dictionary and Handbook, 8th edition, edited by Wenninger and Canterbery (The Cosmetic, Toiletry, and Fragrance Association, Inc., Washington, D.C., 2000), p.1773-1774. Some non-limiting examples of these optional ingredients are given below.

Preferably the compositions of the present invention comprise at least about 5%, more preferably at least about 10%, even more preferably at least about 15%, still more preferably at least about 20% of oil-soluble skin conditioning agent, said conditioning agent preferably being an emollient.

Preferably the emollients for use herein are selected from:

i) Straight and branched chain hydrocarbons having from about 7 to about 40 carbon atoms, such as dodecane, squalane, petrolatum, cholesterol and derivatives thereof, hydrogenated polyisobutylene, isohexadecane and the C₇-C₄₀ isoparaffins, which are C₇-C₄₀ branched hydrocarbons.

ii) C₁-C₃₀ alcohol esters of C₁-C₃₀ carboxylic acids and of C₂-C₃₀ dicarboxylic acids, e.g. isononyl isononanoate, isopropyl myristate, myristyl propionate, isopropyl stearate, behenyl behenate, dioctyl maleate, diisopropyl adipate, and diisopropyl dilinoleate.

iii) mono-, di- and tri-glycerides of C₁-C₃₀ carboxylic acids and ethoxylated derivatives thereof. Suitable polyethylene glycol derivatives of glycerides include PEG-20 almond glycerides, PEG-60 almond glycerides, PEG-11 avocado glycerides, PEG-6 capric/caprylic glycerides, PEG-8 capric/caprylic glycerides, PEG-20 corn glycerides, PEG-60 corn glycerides, PEG-60 evening primose glycerides, PEG-7 glyceryl cocoate, PEG-30 glyceryl cocoate, PEG-40 glyceryl cocoate, PEG-78 glyceryl cocoate, PEG-80 glyceryl cocoate, PEG-12 glyceryl dioleate, PEG-15 glyceryl isostearate, PEG-20 glyceryl isostearate, PEG-30 glyceryl isostearate, PEG-75 cocoa butter glycerides, PEG-20 hydrogenated palm oil glycerides, PEG-70 mango glycerides, PEG-13 mink glycerides, PEG-75 shorea butter glycerides, PEG-10 olive glycerides, PEG-12 palm kernal glycerides, PEG-45 palm kernal glycerides, PEG-8 glyceryl laurate and PEG-30 glyceryl laurate. Mixtures of polyethylene glycol derivatives of glycerides can also be used herein.

iv) alkylene glycol esters of C₁-C₃₀ carboxylic acids, e.g. ethylene glycol mono- and di-esters, and propylene glycol mono- and di-esters of C₁-C₃₀ carboxylic acids e.g., ethylene glycol distearate.

v) Organopolysiloxane oils. The organopolysiloxane oil may be volatile, non-volatile, or a mixture of volatile and non-volatile silicones. The term “non-volatile” as used in this context refers to those silicones that are liquid under ambient conditions and have a flash point (under one atmospheric of pressure) of or greater than about 100° C. The term “volatile” as used in this context refers to all other silicone oils. Suitable organopolysiloxanes can be selected from a wide variety of silicones spanning a broad range of volatilities and viscosities. Non-volatile polysiloxanes are preferred. Suitable silicones are disclosed in U.S. Pat. No. 5,069,897, issued Dec. 3, 1991. Preferred for use herein are organopolysiloxanes selected from polyalkylsiloxanes, alkyl substituted dimethicones, dimethiconols, polyalkylaryl siloxanes, and mixtures thereof. More preferred for use herein are polyalkylsiloxanes and cyclomethicones. Preferred among the polyalkylsiloxanes are dimethicones.

vi) Vegetable oils and hydrogenated vegetable oils. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, partially and fully hydrogenated oils from the foregoing sources, and mixtures thereof.

vii) animal fats and oils, e.g. cod liver oil, lanolin and derivatives thereof such as acetylated lanolin and isopropyl lanolate. Lanolin oil is preferred.

viii) C₄-C₂₀ alkyl ethers of polypropylene glycols, C₁-C₂₀ carboxylic acid esters of polypropylene glycols, and di- C₈-C₃₀ alkyl ethers, examples of which include PPG-14 butyl ether, PPG-15 stearyl ether, dioctyl ether, dodecyl octyl ether, and mixtures thereof.

ix) polyol carboxylic acid esters.

x) PPG Ethers

xi) mixtures of the above.

Preferred emollients for use in the compositions herein are selected from the group consisting of dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof. More preferred emollients for use herein are selected from the group consisting of polyol carboxylic acid esters, PPG Ethers, petrolatum, derivatives thereof and mixtures thereof.

These esters are derived from a sugar or polyol moiety and one or more carboxylic acid moieties. Depending on the constituent acid and sugar, these esters can be in either liquid or solid form at room temperature. Examples of liquid esters include: glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof. Examples of solid esters include: sorbitol hexaester in which the carboxylic acid ester moieties are palmitoleate and arachidate in a 1:2 molar ratio; the octaester of raffinose in which the carboxylic acid ester moieties are linoleate and behenate in a 1:3 molar ratio; the heptaester of maltose wherein the esterifying carboxylic acid moieties are sunflower seed oil fatty acids and lignocerate in a 3:4 molar ratio; the octaester of sucrose wherein the esterifying carboxylic acid moieties are oleate and behenate in a 2:6 molar ratio; and the octaester of sucrose wherein the esterifying carboxylic acid moieties are laurate, linoleate and behenate in a 1:3:4 molar ratio. A preferred solid material is sucrose polyester in which the degree of esterification is 7-8, and in which the fatty acid moieties are C18 mono- and/or di-unsaturated and behenic, in a molar ratio of unsaturates: behenic of 1:7 to 3:5. A particularly preferred solid sugar polyester is the octaester of sucrose in which there are about 7 behenic fatty acid moieties and about 1 oleic acid moiety in the molecule. Other materials include cottonseed oil or soybean oil fatty acid esters of sucrose. The ester materials are further described in, U.S. Pat. No. 2,831,854, U.S. Pat. No. 4,005,196, to Jandacek, issued Jan. 25, 1977; U.S. Pat. No. 4,005,195, to Jandacek, issued Jan. 25, 1977, U.S. Pat. No. 5,306,516, to Letton et al., issued Apr. 26, 1994; U.S. Pat. No. 5,306,515, to Letton et al., issued Apr. 26, 1994; U.S. Pat. No. 5,305,514, to Letton et al., issued Apr. 26, 1994; U.S. Pat. No. 4,797,300, to Jandacek et al., issued Jan. 10, 1989; U.S. Pat. No. 3,963,699, to Rizzi et al, issued Jun. 15, 1976; U.S. Pat. No. 4,518,772, to Volpenhein, issued May 21, 1985; and U.S. Pat. No. 4,517,360, to Volpenhein, issued May 21, 1985.

The polyol fatty acid polyesters suitable for use herein can be prepared by a variety of methods well known to those skilled in the art. These methods include: transesterification of the polyol with methyl, ethyl or glycerol fatty acid esters using a variety of catalysts; acylation of the polyol with a fatty acid chloride; acylation of the polyol with a fatty acid anhydride; and acylation of the polyol with a fatty acid, per se. See, for example, U.S. Pat. No. 2,831,854; U.S. Pat. No. 4,005,196, to Jandacek, issued Jan. 25, 1977.

An especially preferred polyol carboxylic acid ester is known by the INCI name sucrose polycottonseedate.

### Water-Soluble Skin-Conditioning Agent

It is preferred that the compositions of the present invention further contains at least 1% of a water-soluble skin-conditioning agent. Preferred water-soluble skin-conditioning agents are humectants. As used herein the term “humectant” means a substance which provides the skin with water-retention benefits. The compositions of the present invention comprise at least about 1%, preferably at least about 5%, more preferably at least about 10%, even more preferably at least about 15%, still more preferably at least about 20%, by weight of water-soluble skin conditioning agent such as humectant.

Any humectant suitable for use in cosmetic compositions may be used herein. Non-limiting examples of suitable humectants for use in the present invention are described in WO98/22085, WO98/18444 and WO97/01326. Another list of suitable humectant can be found in CTFA International Cosmetic Ingredient Dictionary and Handbook, 8th edition, edited by Wenninger and Canterbery, (The Cosmetic, Toiletry, and Fragrance Association, Inc., Washington, D.C., 2000), p.1768-1773. Some non-limiting examples of these optional ingredients are given below.

Preferably the humectants for use herein are selected from, but not limited to; amino acids and derivatives thereof such as proline and arginine aspartate, 1,3-butylene glycol, propylene glycol and water and codium tomentosum extract, collagen amino acids or peptides, creatinine, diglycerol, biosaccharide gum-1, glucamine salts, glucuronic acid salts, glutamic acid salts, polyethylene glycol ethers of glycerin (e.g. glycereth 20), glycerin, glycerol monopropoxylate, glycogen, hexylene glycol, honey, and extracts or derivatives thereof, hydrogenated starch hydrolysates, hydrolyzed mucopolysaccharides, inositol, keratin amino acids, urea, LAREX A-200 (available from Larex), glycosaminoglycans, methoxy PEG 10, methyl gluceth-10 and -20 (both commercially available from Amerchol located in Edison, N.J.), methyl glucose, 3-methyl-1,3-butanediol, N-acetyl glucosamine salts, polyethylene glycol and derivatives thereof (such as PEG 15 butanediol, PEG 4, PEG 5 pentaerythitol, PEG 6, PEG 8, PEG 9), pentaerythitol, 1,2 pentanediol, PPG-1 glyceryl ether, PPG-9, 2-pyrrolidone-5-carboxylic acid and its salts such as glyceryl pca, saccharide isomerate, SEACARE® (available from Secma), sericin, silk amino acids, sodium acetylhyaluronate, sodium hyaluronate, sodium poly-aspartate, sodium polyglutamate, sorbeth 20, sorbeth 6, sugar and sugar alcohols and derivatives thereof such as glucose, mannose and polyglycerol sorbitol, trehalose, triglycerol, trimethyolpropane, tris (hydroxymethyl) amino methane salts, and yeast extract, and mixtures thereof.

More preferably, the humectants for use herein are selected from the group consisting of glycerin, urea, butylene glycol, polyethylene glycol, propylene glycol, derivatives thereof and mixtures thereof. Even more preferably, the humectants for use herein are selected from the group consisting of glycerin, urea, derivatives thereof and mixtures thereof, especially glycerin.

### Propellant

In a preferred embodiment of the present invention, the mousse is formed by the passage of a pressurized mixture of a concentrate and a propellant through a nozzle. Preferably, the propellant is in the form of a compressed gas, typically a liquefiable gas. The mixture is preferably contained in a dispenser equipped with a dispensing head and valve, and pressurized with the propellant. Upon discharge of the composition through the dispensing head, the volatilization of the dispersed liquid droplets of propellant causes the dispensed concentrate to foam. Depending upon the precise formulation of the concentrate and the propellant, the dispensed product may range from a dense creamy foam to a light foam, dependent on desired aesthetics in the hand and when spread onto the substrate.

In another embodiment, the propellant described above is at least partially housed between a bag comprising the composition and the external pack cavity (“bag-in-a-can”). The propellant which is exterior to the bag is of greater pressure than that which is inside the bag. Total amount of propellant between the interior bag and the exterior container shall be of sufficient pressure and amount to insure complete evacuation of the bag contents. This serves to force the mousse-forming composition out of the bag when the pressure is released. The composition is dispensed as a lotion or a gel usually containing an additional foaming agent. Suitable foaming agents can be selected from liquids with a boiling point between 20° C. and 37° C. in standard conditions. The temperature of the substrate (for example skin) must be sufficiently high to cause said liquids contained in the gel or lotion to boil. The formation of the mousse is accelerated by rubbing the mousse-forming gel or liquid once dispensed onto the substrate, allowing for the dispersed foaming agents to more readily reach their boiling point and flash foam.

In both embodiments, the pressurized package contains from about 0.1% to about 40%, more preferably from about 0.5% to about 20%, even more preferably from about 1% to about 10%, still more preferably of from about 2% to about 6% of propellant, by weight of the total composition. Any propellant suitable for use in cosmetic compositions can be used herein. Non-limiting examples of suitable propellants are nitrous oxide, carbon dioxide, nitrogen, and hydrocarbon propellants such as propane, isobutane, n-butane, isopentane, n-pentane, and dimethyl ether. Preferred propellants are selected from propane, isobutane, n-butane, isopentane, n-pentane, and mixtures thereof. Chlorinated fluorocarbons such as 1,1-difluoro or 1,1,1,2-tetrafluoro ethane are also suitable but their use is being limited for environmental reasons. These propellants usually have a low boiling point and are in a gaseous form at room temperature in standard conditions. Propane, for example, has a boiling point in standard conditions of −41.2° C. and isobutane of −12° C.

### Other Ingredients

The compositions herein can contain a variety of optional components suitable for rendering the present compositions more cosmetically or aesthetically acceptable or to provide them with additional usage benefits. Such conventional optional ingredients are well known to those skilled in the art. These include any cosmetically acceptable ingredients such as those found in the CTFA International Cosmetic Ingredient Dictionary and Handbook, 8th edition, edited by Wenninger and Canterbery, (The Cosmetic, Toiletry, and Fragrance Association, Inc., Washington, D.C., 2000). Some non-limiting examples of these optional ingredients are given below.

Polar Solvent

The compositions of the present invention preferably include a polar solvent. Any polar solvent suitable for use in cosmetic compositions may be used herein. However, the polar solvent must be sufficiently polar to drive the formation of vesicles in the present invention. Preferably the polar solvent used in the compositions of the present invention is water.

Preferably the present compositions will contain from 10% to 90%, more preferably from 20% to 80%, even more preferably from 30% to 60%, by weight, of polar solvent.

Thickeners

The compositions of the present invention preferably contain thickeners. Any thickener suitable for use in cosmetic compositions can be used herein. Preferred thickeners are selected from non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols and mixtures thereof.

It was found that the sensory perception in and after-use of cosmetic compositions comprising a) quaternary ammonium compounds such as those disclosed herein and in particular diester quaternary ammonium agents according to Formula (II) or (III) and b) high level of skin conditioning agent(s) could be significantly improved by using a cationic polymer such as Polyquat 37. This benefit was also found for compositions in the form of creams, lotions, gels, and the like such as those described in WO2001/00170. Example of such a body-lotion in the form of an O/W emulsion comprises 6% N,N-di(canoloyloxyethyl)-N,N-dimethylammonium chloride, 30% glycerin, 15% Sefa Cottonate, 5% Petrolatum and 0.45% Polyquat 37. The emollient system can also contain PPG15 Stearyl Ether.

Other Skin Benefit Agents

Other skin benefit agents may be useful in the compositions of the present invention, for example:

a) Vitamin compounds, particularly vitamin B₃ compounds as described in WO97/39733, which are useful for regulating skin condition.

(b) Anti-Wrinkle and Anti-Skin Atrophy Actives,

(c) Antimicrobial and Antifungal Actives,

(d) Sunscreen Actives.

Examples of such actives can be found in WO2001/00170 p.18-22, incorporated herein by reference.

### Formulation Process

The compositions of the present invention are preferably prepared in such a way that the quaternary ammonium compounds form vesicles. It is also preferred that said vesicles also comprise a skin-conditioning agent such as a emollient and/or humectant. In order to ensure optimal performance characteristics it is preferred that the compositions used in the present invention are prepared as follows:

i) all or part of the quaternary ammonium agent is mixed with humectant (where included), water soluble skin care actives (where included), and, preferably, polar solvent at a temperature which is higher than the melting point of the quaternary ammonium agent;

ii) optionally, the mixture is vigorously agitated;

iii) in a separate vessel the emulsion is prepared as follows; the oil phase containing the emollients, the relevant thickener in case the said thickener is oil soluble, and any remaining quaternary ammonium agent are mixed together at a temperature that is higher than the melting point of the quaternary ammonium compound. The aqueous phase is prepared separately. The water, the relevant thickener in case the said thickener is water soluble, and any remaining water-soluble ingredients are heated to the same temperature as the oil phase.

iv) The temperature of the oil and aqueous phases of the emulsion are then approximately equalized and the aqueous phase is combined with the oil phase with agitation.

v) On production of the emulsion the mixture formed in step (i) is added to the aforementioned emulsion with agitation.

vi) The concentrate is filled in the package (container) of choice. A valve system is added (if included in the components of the pack) and the propellant of choice injected into the can under pressure.

### Method of Use

The cosmetic compositions of the present invention may be used in a conventional manner for the treatment of skin. An effective amount of the composition, typically from about 0.1 grams to about 50 grams, preferably from about 1 gram to about 20 grams, is applied to wet or dry, preferably wet, skin. Application of the composition typically includes working the composition into the skin, generally with the hands and fingers. The composition is then left on the skin or, preferably, the skin is rinsed.

The preferred method of treating the skin, therefore, comprises the steps of:

(a) applying an effective amount of the cosmetic composition to the skin,

(b) rinsing the skin.

A preferred aspect of the present invention involves the above method with an application of the composition on dry skin before an application on wet skin. Therefore, a preferred method comprises:

(i) applying to dry skin an effective amount of the cosmetic composition;

(ii) rinsing the skin under a shower;

(iii) further application of said composition; and

(iv) further rinsing.

Much of the damage to human skin is caused by repeated exposure to surfactant containing compositions during washing routines. It has been found that this damage can be mitigated using the present compositions. Therefore, another preferred method comprises:

(i) washing the skin using a composition comprising surfactants;

(ii) rinsing the skin;

(iii) applying to the wet skin a composition according to the present invention;

(iv) rinsing the skin.

It has also been found that the present compositions are particularly useful when incorporated as part of a regular routine. Therefore, another preferred method comprises:

(i) applying to the skin a composition comprising:

(a) at least one quaternary ammonium compound;

(b) humectant; and

(ii) rinsing the skin;

(iii) repeating steps (i) and (ii) within 48 hours.

The present methods can also be useful in mitigating damage caused by exposure of the skin to ultra violet radiation, damage caused by exposure of the skin to water during swimming or similar water based exercise, damage caused by shaving or exfoliation or damage caused by exposure of the skin to water during bathing.

### EXAMPLES

The following examples further illustrate the preferred embodiments within the scope of the present invention. The examples are given solely for the purposes of illustration and are not to be construed as limitations of the present invention as many variations of the invention are possible without departing from its spirit or scope. Unless otherwise indicated, all ingredients are expressed on a weight percentage of the total composition. The ingredients are mixed and processed according to the process of manufacture described hereinabove.

| Example | 1 | 2 | 3 | 4 | 5 | 6 | 7 | 8 | 9 | 10 |
|---|---|---|---|---|---|---|---|---|---|---|
| | Weight % | | | | | | | | | |
| Quaternary Ammonium | 6 | | 3 | 8 | 8 | 7 | 10 | 3 | 6 | 6 |
| Agent (see notes below) | | | | | | | | | | |
| Glycerin | 32 | 30 | 1 | 1 | 31 | 21 | 10 | 32 | 30 | 29 |
| Petrolatum | 6 | 1 | 30 | 1 | 15 | 10 | 6 | 9 | 10 | 2 |
| Lanolin ¹ | | | | 3 | 2 | 3 | | | | 3 |
| Coronet Lanolin ¹ | | | | 2 | | | | | | 2 |
| Isohexadecane | | | | | 4 | 2 | | | | |
| Super Sterol Esters ¹ | | | | 0.5 | | 2 | | | | 0.5 |
| Sefa Cottonate | 15 | 14 | | 12 | | 3 | 15 | 4 | 5 | |
| PPG 15 Stearyl Ether | | | | | | | | 6 | | 2.5 |
| Dimethicone 10Cst | | | | | 0.1 | | | | | |
| Dimethicone 1000Cst | | | | | | 0.2 | 0.1 | | | |
| Cyclomethicone | | | | | | | | | | 0.1 |
| FlorasunPEG-10 ² | | | | | | | | | 0.1 | |
| Methyl Paraben | 0.3 | 0.2 | 0.3 | 0.2 | 0.3 | 0.2 | 0.3 | 0.2 | 0.3 | |
| Propyl Paraben | 0.2 | 0.2 | 0.2 | 0.2 | 0.2 | 0.2 | 0.2 | 0.2 | 0.2 | |
| Phenoxetol | 0.3 | 0.3 | 0.3 | 0.3 | 0.3 | 0.3 | 0.3 | 0.3 | 0.3 | |
| Niacinamide | | | | | 0.2 | | | | | |
| Panthenol | | | | | 3 | | | | | |
| Vitamin E Acetate | | | | | 1 | 0.5 | | | | |
| Retinol Palmitate | | | | | | | | | 0.1 | |
| Parsol 1789 ³ | | | | | | | | | | 0.5 |
| Parsol MCX ³ | | | | | | | | | | 2.5 |
| Citric acid | | | | | | | | | | 0.5 |
| Salicylic acid | | | | | | 0.5 | | | | |
| Triethanolamine | | | 0.1 | | 0.1 | | 0.1 | | 0.1 | |
| Sodium Hydroxide | 0.1 | 0.2 | | 0.2 | | 0.4 | | 0.2 | | 0.2 |
| Polyguaternium 10 | 0.15 | 0.1 | | | | | | 0.1 | 0.5 | |
| Polyguaternium 37 | 0.45 | | | 0.1 | | | | | | |
| Polymer KG 30 ⁴ | | | | | | | | | | 0.1 |
| Fragrance | 1 | 0.8 | 1 | 1 | 1 | 1 | 1 | | 1 | 1 |
| Cetyl Alcohol | 1 | 1 | 0.5 | 0.75 | 0.6 | 0.8 | 0.5 | 0.5 | 1 | 0.5 |
| Stearyl Alcohol | 1.3 | 1.1 | 0.5 | 0.9 | | 0.2 | | | | 0.3 |
| Cetearyl Alcohol | | | 0.2 | | | | | | | |
| Behenyl Alcohol | 0.35 | 0.4 | | 0.3 | 0.1 | | | | | 0.3 |
| Stearic Acid | 0.25 | | | | | | | | | |
| Steareth 2 | | | | | 1.6 | | | | | |
| Steareth 21 | | | | | 0.4 | | | | | |
| Natrosol 330 Plus | | | 0.25 | | | | 0.1 | | | |
| Natrosol 250 H | | 0.3 | | | | | | 0.1 | | |
| Jaguar HP 105 ⁵ | | | | | | | | | | 0.1 |
| Jaguar C14S ⁵ | | | | 0.1 | | | | | | |
| Xanthan Gum | | | | | | | | | 0.2 | |
| Sodium Chloride | | | | 0.1 | | 0.1 | | 0.1 | | 0.1 |
| Propellant (see notes) | 3 | 3 | 6 | 4 | 7 | 6 | 3 | 4 | 3 | 7 |
| Isopentane (Foaming | | | | | | 3 | | | | |
| agent) | | | | | | | | | | |
| Water | qs | qs | qs | qs | qs | qs | qs | qs | qs | qs |

¹Available from Croda
²Available from Floratech, AZ, USA
³Available from Hoffman La Roche, NJ, USA
⁴Available from Amerchol, NJ, USA
⁵Available from Rhodia, NJ, USA

Quaternary Ammonium Notes

In Examples 1-4 the quaternary ammonium compound used is Dicanoloyoxylethyl hydroxyethylmonium Methyl Sulfate supplied by Goldschmidt, trade name Rewoquat V3620/Rewoquat V10028 (Developmental Material)

In Example 5 the quaternary ammonium compound used is Distearyl Dimethyl Ammonium Chloride supplied by Goldschmidt, trade name Varisoft TA 100.

In Example 6 the quaternary ammonium compound used is Methylbis(hydrogenated tallowamidoethyl) (2-hydroxyethyl)ammonium methyl sulfate supplied by Goldschmidt, trade name Varisoft 110.

In Example 7 the quaternary ammonium compound used is Methylbis(tallowamidoethyl)(2-hydroxyethyl)ammonium methyl sulfate supplied by Goldschmidt, trade name Varisoft 222.

In Example 8 the quaternary ammonium compound used is Methyl-1-tallowamidoethyl-2-tallowimidazolinium methyl sulfate supplied by Goldschmidt, trade name Varisoft 475, Varisoft 445

In Example 9 and 10 the quaternary ammonium compound used is Dihydrogenated palmoylethyl hydroxyethylmonium methosulfate supplied by Goldschmidt, trade name Rewoquat WE28

Propellant Notes

The percentage of propellant is expressed by weight of the total composition, which is the sum of the weight of the propellant and the concentrate.

In examples 1,2,5 the propellant used is Aeron® A-70 (Mixture of propane, isobutane, n-butane), supplied by diversified CPC International.

In examples 3, 9 the propellant used is Aeron® A-46 (Mixture of propane, isobutane, n-butane), supplied by diversified CPC International.

In example 4, the propellant used is Aeron® A-31 (Mixture of propane, isobutane, n-butane), supplied by diversified CPC International.

In example 6 the propellant (Aeron® A-70) is housed between a bag comprising the concentrate and the external pack cavity (“bag-in-a-can”). The other examples could also be expressed as finished product delivered via bag in can mechanics using evacuation propellants of greater pressure than resistant bag pressure created by the composition of the bag and the internal pressure of foaming agents contained in the mixture. In example 7 the propellant used is Aeron®) A-108 (Mixture of propane, isobutane, n-butane), supplied by diversified CPC International.

In example 8, the propellant used is Nitrous Oxide.

In example 10, the propellant used is Carbon Dioxide.

What is claimed is:

1. A composition comprising, by weight of said composition:
(i) at least about 0.1% of a quaternary ammonium agent; and
(ii) at least about 1% of an oil-soluble skin-conditioning agent;
wherein said composition is in the form of a mousse.

2. A composition according to claim 1 comprising, by weight of said composition, at least about 5% of said oil-soluble skin-conditioning agent.

3. A composition according to claim 2 wherein said oil-soluble skin-conditioning agent is selected from the group consisting of dodecane, squalane, cholesterol, isohexadecane, isononyl isononanoate, polypropylene glycol ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

4. A composition according to claim 1 further comprising at least about 1%, by weight of said composition, of water-soluble skin-conditioning agent.

5. A composition according to claim 4 comprising, by weight of said composition, at least about 5% of said water-soluble skin-conditioning agent.

6. A composition according to claim 5, wherein said water-soluble conditioning agent is selected from the group consisting of glycerin, urea, butylene glycol, polyethylene glycol, propylene glycol, derivatives thereof and mixtures thereof.

7. A composition according to claim 1 comprising at least about 0.5%, by weight of said composition, of said quaternary ammonium agent.

8. A composition according to claim 7, wherein said quaternary ammonium agent forms vesicles.

9. A composition according to claim 8 wherein said quaternary ammonium agent is selected from the group consisting of general formula (II) below, general formula (III) below, and mixtures thereof: 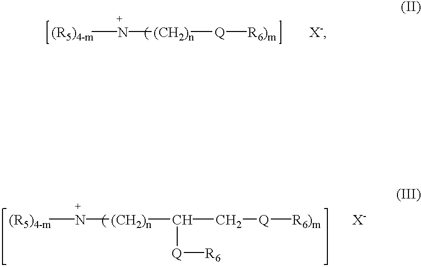
wherein each R₅ unit is independently selected from hydrogen, branched or straight chain C₁-C₆ alkyl, branched or straight chain C₁-C₆ hydroxyalkyl and mixtures thereof; wherein each R₆ unit is independently selected from the group consisting of independently linear or branched C₁₁-C₂₂ alkyl, linear or branched C₁₁-C₂₂ alkenyl, and mixtures thereof; wherein X⁻ is an anion which is compatible with skin care actives and adjunct ingredients; wherein m is an integer from 1 to 4; wherein n is an integer from 1 to 4, and Q is a carbonyl unit selected from the group consisting of 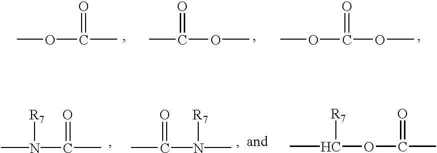
wherein R₇ is selected from the group consisting of hydrogen, C₁-C₄ alkyl, C₁-C₄ hydroxyalkyl, and mixtures thereof.

10. A composition according to claim 9 wherein the quaternary ammonium agent is selected from the group consisting of N,N-di(canoloyloxyethyl)-N,N-dimethyl ammonium chloride, N,N-di(canoloyloxyethyl)-N-methyl-N-(2-hydroxyethyl) ammonium methyl sulfate, N,N-di(canoloyloxyethyl)-N-methyl-N-(2-hydroxyethyl) ammonium chloride and mixtures thereof.

11. A composition according to claim 1 wherein said composition is substantially free of anionic surfactant.

12. A composition according to claim 1 wherein said composition is substantially free of non-ionic surfactant.

13. A first composition packaged in a pressurized container which forms upon discharge from said container a second composition comprising, by weight of said second composition:
(i) at least about 0.1% of a quaternary ammonium agent; and
(ii) at least about 1% of an oil-soluble skin-conditioning agent;
wherein said composition is in the form of a mousse.

14. A composition according to claim 13, wherein said container comprises a nozzle, and said first composition is discharged through said nozzle.

15. A composition according to claim 14 further comprising a propellant.

16. A composition according to claim 15, wherein said first composition comprises, by weight of the first composition, from about 0.1% to about 40% of said propellant.

17. A method of treating skin comprising the subsequent steps of:
(i) washing the skin using a composition comprising surfactants;
(ii) rinsing the skin;
(iii) applying to the wet skin a composition according to claim 1; and
(iv) rinsing the skin.
